(19) United States
(12) Patent Application Publication
(10) Pub. No.: US 2006/0128647 A1
(43) Pub. Date: Jun. 15, 2006

(54) Novel oligonucleotides and treating cardiac disorders by using the same

(76) Inventors: Daniel Sigg, St. Paul, MN (US); Michael Ujhelyi, Maple Grove, MN (US); James A. Coles JR., Columbia, MD (US); Rudolfo Padua, Richfield, MN (US)

Correspondence Address: CAROL M. NIELSEN;WINSTEAD SECHREST & MINICK, P.C., 2400 BANK ONE CENTER, 910 TRAVIS STREET, HOUSTON, TX, 77002, US

(21) Appl. No.: 11/018,443

(22) Filed: Dec. 21, 2004

Related U.S. Application Data
(60) Provisional application No. 60/532,846, filed on Dec. 24, 2003.

(51) Int. Cl. A61K 48/00; A61K 38/17; C07H 21/04; C12N 5/08; C12N 15/87

(52) U.S. Cl. 514/44; 514/12; 536/23.2; 435/366; 435/455

(57) ABSTRACT

Novel oligonucleotides and methods of treating a cardiac disease or disorder using the same are provided. The oligonucleotides are useful in modulating the expression of the connexin 43 protein and may be combined with other biologically active agents and compositions to treat cardiac disease. Methods of modulating connexin expression include the suppression of the expression of connexin 43 and the inducement of expression of connexin 45.

### CROSS-REFERENCES TO RELATED APPLICATIONS

The present application claims priority under Title 35, United States Code, § 119 to provisional application U.S. Pat. App. Ser. No. 60/532,846 filed Dec. 24, 2003.

### FIELD OF INVENTION

The present invention relates generally to methods of using novel oligonucleotides for providing pacemaker function, and more specifically to novel oligonucleotides, constructs and cell transfected cell lines useful in modulating connexin 43.

### BACKGROUND OF INVENTION

The mammalian heart maintains an intrinsic rhythm by creating electric stimuli. Generally, the stimuli form a depolarization wave that propagates within specialized cardiac conducting tissue and the myocardium. The usually well-ordered wave movement facilitates coordinated contractions of the myocardium. These contractions are the engine that moves blood throughout the body. See, generally, The Heart and Cardiovascular System. Scientific Foundations (1986) Fozzard, H. A. et al. eds, Raven Press, NY.

Cardiac electrophysiology has been the subject of intense interest. Generally, the cellular basis for all cardiac electrical activity is the action potential (AP). The AP is conventionally divided into five phases in which each phase is defined by the cellular membrane potential and the activity of potassium, chloride, sodium and calcium ion channel proteins that affect that potential. Propagation of the AP throughout the heart is known to involve gap junctions. See e.g., Tomaselli et al., Cardiovasc. Res. (1999) 42:270 and references cited therein.

Under most circumstances, cardiac stimuli are controlled by recognized physiological mechanisms. Cardiac myogenic tissue is directly effected by both the electrical pulse and the contraction of the tissue. For example, studies have shown that electrical stimulation of quiescent neonatal cardiac myocytes causes hypertrophy in vitro and activates hypertrophy-associated pathways and certain marker genes. See e.g., McDonough et al., J. Biol. Chem. (1997) 272:24046-14053; McDonough et al., J. Biol. Chem. (1992) 267:11665-11668; Xia et al., J. Biol. Chem. (1998) 273:12593-12598; Xia et al., Proc. Natl. Acad. Sci. (1997) 94:11399-11404; Xia et al., J. Biol. Chem. (2000) 275:1855-1863. Likewise, the ANP and adenylosuccinate synthetase 1 (Adss1) genes are strongly induced by electrical pacing, and their respective promoters can be activated by 10-fold or more by electrical stimulation of myocytes. See e.g., Xia et al., J. Biol. Chem. (1998) 273:12593-12598; Xia et al., J. Biol. Chem. (2000) 275:1855-1863. Cardiac myocytes exhibit transient increases in cytoplasmic calcium that serve as the driving force behind contraction.

In the upper part of the right atrium of the heart is a specialized bundle of myocardial cells known as the sinoatrial node (SA node). Acting as the heart's natural pacemaker the SA node “fires” at regular intervals to cause the heart beat with a rhythm of about 60 to 70 beats per minute for a healthy, resting heart. The electrical impulse from the SA node triggers a sequence of electrical events in the heart to control the orderly sequence of muscle contractions that pump the blood out of the heart.

The SA node has distinct electrophysiological properties. In particular, the conduction velocity within SA node can be as low as 0.03 meters/second, and the low conductance is mostly due to decreased intercellular coupling due to a lack of connexin 43, and presence of connexin 45. The decreased conduction velocity of the SA node is mostly due to a differential expression of the various isoforms of the connexin proteins.

Connexin proteins are a family of homologous proteins found in connexins of gap junctions as homo- or heterohexameric arrays. Connexins are pore-like complex protein structures forming channels (otherwise known as gap junctions) between cells. Each cell contributes one hemi-channel to form a connexin. Indeed, connexin proteins are the major gap junction protein involved in the electrical coupling of myocardial cells. Gap junctions regulate intercellular passage of molecules, including inorganic ions and second messengers, thus achieving electrical coupling of cells. Over 15 connexin subunit isoforms are known, varying in size between about 25 kDa and 60 kDa and generally having four putative transmembrane .quadrature.-helical spanners. Different connexins are specific for various parts of the heart.

Connexin proteins found in the cardiovascular system include connexin 37 (“Cx37”), connexin 40 (“Cx40”), connexin 43 (“Cx43”), and connexin 45 (“Cx45”) See, Van Veen, A A; Van Rijen, H V; Opthof, T., Cardiovascular Research 2001 Aug. 1, 51(2):217-29.; Severs, N J; Rothery, S; Dupont, E; Coppen, S R; Yeh, H I; Ko, Y S; Matsushita, T; Kaba, R; Halliday, D., Microscopy Research and Technique 2001 Feb. 1, 52(3):301-22; Kwong, K F; Schuessler, R B; Green, K G; Laing, J G; Beyer, E C; Boineau, J P; Saffitz, J E., Circulation Research 1998 Mar. 23, 82(5):604-12).

The primary connexin isoforms found in the heart are connexin 40, 43 and 45. Each connexin isoform has distinct conductances. For example, connexin 43 has a conductance of 100 pS. Connexin 40 has a conductance of 180 pS. Connexin 45 has a low conductance of only 25 pS. Connexin 43 is not expressed in the SA node.

Other parts of the heart also utilize the connexin proteins as a gap junction protein involved in the electrical coupling of cells. In the crista terminalis, a part of the normal right atrium of the heart, the predominant connexin isoform in the atrium is connexin 43. Here, the conduction velocities may be as high as 1.2 m/sec and connexin 43 is believed to account for the increased conduction velocities. Moreover, in the Purkinje fiber system, conduction velocities are even higher (2.4 m/s), and the predominant connexin isoform found is connexin 40.

Currents across gap junctions are also regulated and gated by a variety of factors, such as pH, voltage, intracellular calcium and phosphorylation. Indeed, even the intercellular coupling of other ion channels, such as sodium channels, effect conduction velocities. However, the role of connexins is key to conductivity of electrical pulses in the heart and the amount of connexin produced is central to regulation of electrical stimulation of the myocytes.

There has been a long-standing recognition that abnormalities of excitable cardiac tissue can lead to abnormalities of the heart rhythm. These abnormalities are generally referred to as arrhythmias. Most arrhythmias are believed to stem from defects in cardiac impulse generation or propagation that can substantially compromise homeostasis, leading to substantial patient discomfort or even death. For example, cardiac arrhythmias that cause the heart to beat too slowly are known as bradycardia, or bradyarrhythmia. In contrast, arrhythmias that cause the heart to beat too fast are referred to as tachycardia, or tachyarrhythmia. See, generally, Cardiovascular Arrhythmias (1973) Dreifus, L. S. and Likoff, W. eds, Grune & Stratton, NY.

The significance of heart related disorders cannot be exaggerated. In the United States alone, cardiac arrest accounts for 220,000 deaths per year. This is thought to account for more than 10% of total American deaths.

Symptoms related to arrhythmias range from nuisance, extra heart beats, to life-threatening loss of consciousness and death. Complete circulatory collapse has also been reported. Morbidity and mortality from such problems continues to be substantial. Atrial fibrillation, a specific form of cardiac arrhythmia, impacts more than 2 million people in the United States. Other arrhythmias account for thousands of emergency room visits and hospital admissions each year. See, e.g., Bosch et al., Cardiovas Res. (1999) 44:121 and references cited therein.

Attempts to treat cardiac arrhythmias and related heart disorders have been largely confined to pharmacotherapy, radiofrequency ablation, use of implantable devices, and related approaches. In particular, radiofrequency ablation has been reported to address a limited number of arrhythmias, e.g., atrioventricular (AV) node re-entry tachycardia, accessory pathway-mediated tachycardia, and atrial flutter. More problematic arrhythmias such as atrial fibrillation and infarct-related ventricular tachycardia, however, are less amenable to this and related therapies. Device-based therapies (pacemakers and defibrillators, for instance) have been reported to be helpful for some patients with bradyarrhythmias and lifesaving for patients with tachyarrhythmias.

There are drugs that regulate arrhythmic events. However, often the effects of a drug are poorly tolerated. Moreover, certain drugs are known to those skilled in the art to carry the risk of mortality. See e.g., Bigger et al., The Pharmacological Basis of Therapeutics 8^{th} supl. ed. (Gilman et al., Eds) McGraw-Hill, NY (1993) and references cited therein.

Recently, genetically altered myocytes (atrial and ventricular) have been shown to function in animals. However, prior art transfected mammalian non-SA nodal cells to alter the ion channel function that supports spontaneous depolarization and pacemaker activity. See Miake et al., Nature (2002) 419:132; U.S. Pat. No. 6,214,620; United States Patent Application Publication Nos. US200210155101A1 and US2003/0104568A1; and PCT Publication Nos. WO 02/087419 and WO 02/098286. However, there is concern about the critical mass of cells needed to express the transfected ion channel gene. Without such a critical mass, there will not be enough current density to drive the entire atrium or ventricle. In addition, intercellular heterogeneity of the ion channels may give rise to multiple and competing pacemaker regions within the atrial or ventricle and/or provide insufficient pacemaker current density due to dispersion in refractoriness. If premature impulses are provided during the cardiac cycle, proarrhythmia may result.

### SUMMARY OF INVENTION

Novel oligonucleotides and methods of using the same are provided. The oligonucleotides are useful in modulating the expression of the connexin 43 protein and may be combined with other biologically active agents and compositions to treat cardiac disease.

The present invention provides methods for therapeutically correcting arrhythmias and other heart disorders using the oligonucleotides that suppress the expression of connexin 43 alone or in combination with other biologically active agents, therapies and/or devices. The expression of connexin 43 (Cx43) may be suppressed while inducing the expression of another protein such as connexin 45 (Cx45). Under optimal conditions, conduction velocities mimic the ones observed in the SA node (e.g., ˜0.03 m/sec).

The methods of the subject invention may be used in conjunction with a fluid delivery catheter and/or other suitable device to introduce genetic material locally to desired regions or a traditional pacemaker device.

### BRIEF DESCRIPTION OF DRAWINGS

For a more complete understanding of the present invention, and the advantages thereof, reference is now made to the following description taken in conjunction with the accompanying drawing, in which:

FIG. 1 is a diagram of a human heart.

FIG. 2 is schematic diagram of a physiological gradient model of conduction velocity in the SA node.

FIG. 3 depicts in silico modeling data demonstrating the influence of gap junction conductance on current density stimulation thresholds and conduction velocity.

FIGS. 4 A-C depict (A) normal Cx distribution in A or V, (B) the Cx43 suppression (via siRNA or mutant Cx43 gene or other means), and (C) the Cx45 overexpression at relatively low levels.

FIGS. 5 A-C depict three examples of viral vector expression cassettes: (A) AAV construct, (B) retroviral construct, and (C) adenoviral construct (shuttle plasmid).

FIG. 6 is a schematic diagram of a right side of a heart, similar to FIG. 1, in which a guiding catheter is positioned for delivery of the genetic construct of the invention.

### DETAILED DESCRIPTION OF THE INVENTION

The present invention provides methods for therapeutically correcting arrhythmias and other heart disorders using novel oligonucleotides that modulate the expression of connexin 43 alone or in combination with other biologically active agents, therapies and/or devices. The compositions and associated methods of the subject invention can suppress the expression of connexin 43 (Cx43) while inducing the expression of connexin 45 (Cx45). Under optimal conditions, conduction velocities mimic the ones observed in the SA node (e.g., ˜0.03 m/sec). The methods of the subject invention may be used in conjunction with a fluid delivery catheter to introduce genetic material locally to desired regions or a traditional pacemaker device.

The methods of the subject invention utilize oligonucleotides and nucleic acid pharmaceutical compositions that interfere with the expression of connexin 43. The claimed oligonucleotides are novel small interfering RNA (“siRNA”). The oligonucleotides are designed to elicit strong and specific suppression of connexin 43 gene expression in different mammalian cell lines.

RNA interference is a post-transcriptional process triggered by the introduction of double-stranded RNA which leads to gene silencing in a sequence-specific manner. RNA interference reportedly occurs naturally in organisms as diverse as nematodes, trypanosmes, plants and fungi. It is believed to protect organisms from viruses, modulate transposon activity and eliminates aberrant transcription products. RNA interference is an important method for analyzing gene function in eukaryotes and as used in the subject invention has been developed as a therapeutic method of gene silencing for heart disease.

Small interfering RNA or siRNA, typically 19-23 base pair, double-stranded RNA of synthetic origin are designed to be specific for the nucleotide sequence of its intended target in mRNA. The siRNA interacts with helicase and nuclease to form a complex termed “RNA-induced silencing complex (RISC).” RISC then unwinds the double-stranded siRNA. Antisense then binds to target RNA, which is then cleaved by RISC. The target RNA is further degraded by cellular nucleases. This process is known as RNA interference or RNAi.

The effectiveness of siRNAs of the most potent siRNAs result in greater than 90% reduction in target RNA and protein levels. See e.g., Caplen et al., Proc. Natl. Acad. Sci. USA (2001) 98: 9746-9747; Elbashir et al., Nature (2001) 411: 494-498; Holen et al., Nucleic Acids Research (2002) 30(8):1757-1766. Certain proven siRNAs that have been shown to be very effective contain 21 bp dsRNAs with 2 short 3′ overhangs. See Table 1 below. The effectiveness of the siRNA depends on structure and position. See Brown et al., TechNotes (2002) 9(1): 3-5; Holen et al., Nucleic Acids Research (2002) 30(8):1757-1766; Jarvis et al., TechNotes (2001) 8(5):3-5.

While the terms used herein are well known to those of skill in the art, the following definitions are provided to facilitate an understanding of the invention:

The term “polynucleotide” refers to a molecule having two or more nucleotides such as an oligonucleotide and fragments or portions thereof, as well as to peptide nucleic acids (PNA), fragments, portions or antisense molecules thereof, and to DNA or RNA of genomic or synthetic origin which can be single- or double-stranded, and represent the sense or antisense strand.

DNA or deoxyribonucleic acid has deoxyribose as the sugar group and nitrogenous bases: adenine (A), guanine (G), thymine (T), and cytosine (C). A single strand of DNA has a sequence of bases A, G, T, and C.

RNA or ribonucleic acid has ribose as the sugar group, and the nucleotide bases: adenine (A), guanine (G), uracil (U), and cytosine (C).

The term “messenger RNA” or “mRNA” refers to RNA that serves as a template for protein synthesis.

The term “gene” refers to a hereditary unit consisting of a sequence of DNA that occupies a specific location on a chromosome and can include regions preceding and following coding DNA as well as introns and exons.

The term “gene expression” refers to conversion of information encoded in a gene first into messenger RNA (“mRNA”) and then to a protein. As used herein, expressed genes may include DNA transcribed into mRNA, but not translated into protein (e.g., transfer and ribosomal RNAs). Gene suppression or silencing is an attenuation of gene expression.

The term “antisense” refers to a strand of DNA molecule whose sequence is complementary to the strand represented in mRNA. The term also refers to an RNA molecule complementary to the strand normally processed into mRNA and translated to a protein. Antisense RNA hybridizes to mRNA, physically blocks mRNA translation and targets the mRNA for destruction by cellular nucleases.

The term “ribozyme” refers to RNA molecules which act as enzymes; that is, possess catalytic activity and can specifically cleave (cut) other RNA molecules.

The term “coding sequence” refers to a nucleic acid sequence of DNA that is transcribed into RNA or a nucleic acid sequence of mRNA translated into a polypeptide, in vitro or in vivo.

The term “ion channel protein” refers to a protein that transports ions across cell membranes.

The term “plasmid” refers to a circular DNA molecule typically found in bacteria.

The term “cDNA” refers to nucleic acids that share the arrangement of sequence elements found in native mature mRNA species, where sequence elements are exons (sequences encoding open reading frames of the encoded polypeptide) and 3′ and 5′ non-coding regions. Normally mRNA species have contiguous exons, with the intervening introns removed by nuclear RNA splicing, to create a continuous open reading frame encoding the polypeptide of interest.

The term “construct” is used interchangeably herein with the terms “vector” and “plasmid” and refers to a DNA molecule that is used to deliver a specific gene(s) into a target cell.

The term “promoter” refers to the nucleotides that direct transcription in a cell. “Promoter” is also meant to encompass those elements sufficient for promoter-dependent gene expression controllable for cell-type specific, tissue-specific or inducible by external signals or agents.

The term “Enhancer” or “Enhancer Element” refers to DNA sequences that increase transcription of genes. Enhancers can usually function in either 3′ to 5′ or 5′ to 3′ orientation and at various distances from a promoter.

The term “PCR,” or “polymerase chain reaction,” refers to a system for in vitro amplification of DNA wherein two synthetic oligonucleotide primers, which are complimentary to two regions of the target DNA (one for each strand) to be amplified, are added to the taget DNA in the presence of excess deoxynucleotides and Taq polymerase, a heat stable DNA polymerase. In a series of temperature cycles, the DNA is repeatedly denatured, annealed to the primers, and a daughter strand extended from the primers. As the daughter strands act as templates in subsequent cycles, amplification occurs in an exponential fashion.

The term “gap junction” refers to small pore-like structures that connect cardiac muscle cells to each other.

The terms “transformation” “refers to a permanent or transient genetic change, typically a permanent genetic change, induced in a cell following incorporation of new nucleic acid (e.g., DNA or RNA exogenous to the cell). Genetic change can be accomplished either by incorporation of the new nucleic acid into the genome of the host cell, or by transient or stable maintenance of the new DNA as an episomal element.

The term “transformed cell,” “transfected cell,” “transduced cell” or “recombinant cell” refers to a cell or an ancestor of a cell into which a DNA molecule has been introduced or incorporated by means of recombinant DNA techniques.

A “vector” is a DNA molecule that is used to carry a specific gene into a target cell.

An “expression vector” is a type of vector that once is inside the cell, the protein that is coded for by the gene is produced by normal transcription and translation process of the host cell. Expression vectors are used for molecular biology techniques. Expression vectors are often specifically designed to contain non-protein coding sequences that act as enhancers and promoter regions and allow for efficient transcription of the gene that is carried on the expression vector.

In gene therapy, a “vector” is vehicle for delivery genetic material such as DNA to a cell. For example, a virus itself may serve as a vector, if it has been re-engineered and is often used to delivery a gene to its target cell.

“AAV” is an adeno-associated virus vector. These viruses cause no known disease in humans, hold long-term expression, and theoretically integrate at specific sites.

“AdV” is an adenovirus vector.

“Lentivirus” is a virus, such as HIV, that incorporates its passenger genes into non-dividing cells.

The term “electrical coupling” refers to the interaction between cells which allow for intracellular communication between cells so as to provide for electrical conduction between the cells. Electrical coupling in vivo provides the basis for, and is generally accompanied by, electromechanical coupling, in which electrical excitation of cells through gap junctions in the muscle leads to muscle contraction.

The term “liposome” refers to a synthetic lipid bilayer vesicle that fuses with a cell's outer membrane and is used to transport molecules into cells.

The term “connexin protein” refers to a protein, or mutant thereof, of a family of homologous proteins found in connexins of gap junctions as homo- or heterohexameric arrays. Connexin proteins are the major gap junction protein involved in the electrical coupling of cells. Gap junctions regulate intercellular passage of molecules, including inorganic ions and second messengers, thus achieving electrical coupling of cells.

The terms “connexin 43” and “Cx43” refer to the amino acid sequences of an isolated the connexin 43 polypeptide, having structural, regulatory, or biochemical functions associated with gap junctions and electromechanical coupling, as obtained from any species, particularly mammalian, including human, rodenti (e.g., murine or rat), bovine, ovine, porcine, murine, or equine, and may be natural, synthetic, semi-synthetic or recombinant, and is meant to include all naturally-occurring allelic variants, and is not meant to limit the amino acid sequence to the complete, native amino acid sequence associated with the recited protein molecule. Cx43 encompasses biologically active Cx43 fragments. Examples of Cx43 include human Cx43 (Genbank Accession Nos. XP.sub.--027460, XP.sub.--027459, XP.sub.--004121, P17302, AAD37802, A35853, NP.sub.--000156, AF151980, M65188, and AAA52131), mouse Cx43 (Genbank Accession Nos. P23242, P18246, A39802, A36623, NP.sub.--034418, NM.sub.--012567, NM.sub.--010288, CAA44640) and rat Cx43 are found at Genbank Accession Nos. P08050, S00532, NP.sub.--036699, AAA75194 and 1404339A.

In a normal human heart, for example, cardiac contraction is initiated by the spontaneous excitation of the sinoatrial (SA) node that is located in the right atrium. The electrical current generated by the SA node travels to the atrioventricular (AV) node where it is then transmitted to the bundle of His and Purkinje network, which branches in many directions to facilitate simultaneous contraction of the left and right ventricles.

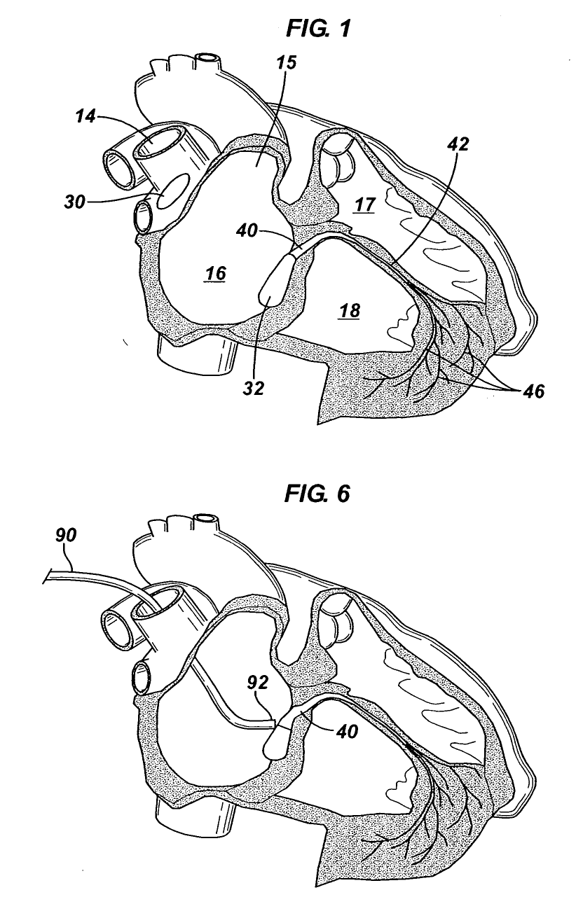

FIG. 1 is a schematic diagram of a right side of a heart having an anterior-lateral wall peeled back to expose a portion of a heart's intrinsic conduction system and chambers of a right atrium (RA) 16 and a right ventricle (RV) 18. Pertinent elements of the heart's intrinsic conduction system, illustrated, in FIG. 1, include a sinoatrial (SA) node 30, an atrioventricular (AV) node 32, a bundle of His 40, a right bundle branch 42, left bundle branches (not shown) and Purkinje fibers 46. SA node 30 is shown at a junction between a superior vena cava 14 and RA 16. An electrical impulse initiated at SA node 30 travels rapidly through RA 16 and a left atrium (not shown) to AV node 32. At AV node 32, the impulse slows to create a delay before passing on through a bundle of His 40, which branches, in an interventricular septum 17, into a right bundle branch 42 and a left bundle branch (not shown) and then, apically, into Purkinje fibers 46. Following the delay, the impulse travels rapidly throughout RV 18 and a left ventricle (not shown). Flow of the electrical impulse described herein creates an orderly sequence of atrial and ventricular contraction to efficiently pump blood through the heart. When a portion of the heart's intrinsic conduction system becomes dysfunctional, efficient pumping is compromised.

Typically, a patient whose SA node 30 has become dysfunctional, may have an implantable pacemaker system implanted wherein lead electrodes are placed in an atrial appendage 15. The lead electrodes stimulate RA 16 downstream of dysfunctional SA node 30 and the stimulating pulse travels on to AV node 32, bundle of His 40, and Purkinje fibers 46 to restore physiological contraction of the heart. However, if a patient has a dysfunctional AV node 32, pacing in atrial appendage 15 will not be effective, since it is upstream of a block caused by the damage.

Pacing at the bundle of His 40 provides the advantage of utilizing the normal conduction system of the heart to carry out ventricular depolarizations. In other words, stimulation provided at the bundle of His will propagate rapidly to the entire heart via the right bundle 42, the left bundle (not shown), and the Purkinje fibers. This provides synchronized and efficient ventricular contraction, unlike pacing from the apex of the right ventricle where the electrical activity propagates at a slower rate because myocardial tissue is a slow conductor compared to the rapidly conducting Purkinje network.

Like cells of other excitable tissue in the body, cardiac cells allow a controlled flow of ions across the membranes. This ion movement across the cell membrane results in changes in transmembrane potential (depolarization), which is a trigger for cell contraction. The heart cells are categorized into several cell types (e.g. atrial, ventricular, etc.) and each cell type has its own characteristic variation in membrane potential. For example, ventricular cells have a resting potential of ˜85 mV. In response to an incoming depolarization wave front, these cells fire an action potential with a peak value of ˜20 mV and then begin to repolarize, which takes ˜350 ms to complete. In contrast, SA nodal cells do not have a stable resting potential and instead begin to spontaneously depolarize when their membrane potential reaches ˜−50 mV. Cells, such as SA nodal cells, that do not have a stable resting transmembrane potential, but instead increase spontaneously to the threshold value, causing regenerative, repetitive phase 4 depolarization, are said to have automaticity.

Cardiac muscle cells are structurally connected to each other via small pore-like structures known as gap junctions, so that when a few cardiac cells depolarize, they act as a current source to adjacent cells causing them to depolarize as well; and these cells in turn relay the electrical charge to adjacent cells. Once depolarization begins within a mass of cardiac cells, it spreads rapidly by cell-to-cell conduction until the entire mass is depolarized causing a mass of cardiac cells to contract as a unit.

The cells in the SA node are specialized pacemaker cells and have the highest firing rate. Depolarization from these cells spreads across the atria. Since atrial muscle cells are not connected intimately with ventricular muscle cells, conduction does not spread directly to the ventricle. Instead, atrial depolarization enters the AV node, and after a brief delay, is passed on to the ventricles via the bundle of His and Purkinje network, initiating cellular depolarization along the endocardiuim. Depolarization then spreads by cell-to-cell conduction throughout the entire ventricular mass.

The SA node's unique cells include a combination of ion channels that endow it with its automaticity. A review of the features of cardiac electrical function and description of the current understanding of the ionic and molecular basis, thereof, can be found in Schram et al., Circulation Research (2002) May 17, pp. 939-950.

Some of the unique features of the SA node cells include the absence of Na⁺ and inwardly rectifying K⁺ (I_{Kl}) channels. In the absence of sodium current, the upstroke of SA node action potential is primarily mediated by L-type Ca²⁺ channels (I_{CaL}). SA node cells do not have a stable resting potential because of the lack of the I_{Kl} and begin to depolarize immediately after the repolarization phase is complete. The maximum diastolic potential for SA node cells is approximately −50 mV compared to −78 mV and −85 mV for atrial and ventricular cells, respectively. The slow depolarization phase is mediated by activation of I_{f} and T-type Ca²⁺ channels and deactivation of slow and rapid potassium (I_{Ks} and I_{Kr}, respectively). The rate of pacemaker discharge in the SA node in a normally functioning heart is approximately in the range of about 60 to 100 beats per minute.

The SA node has distinct electrophysiological properties. In particular, the conduction velocity within the SA node is as low as 0.03 meters/second, and the low intercellular conductance in the SA node is mostly due to decreased intercellular coupling due to a lack of connexin 43, and presence of connexin 45. It is therefore important that this balance be maintained.

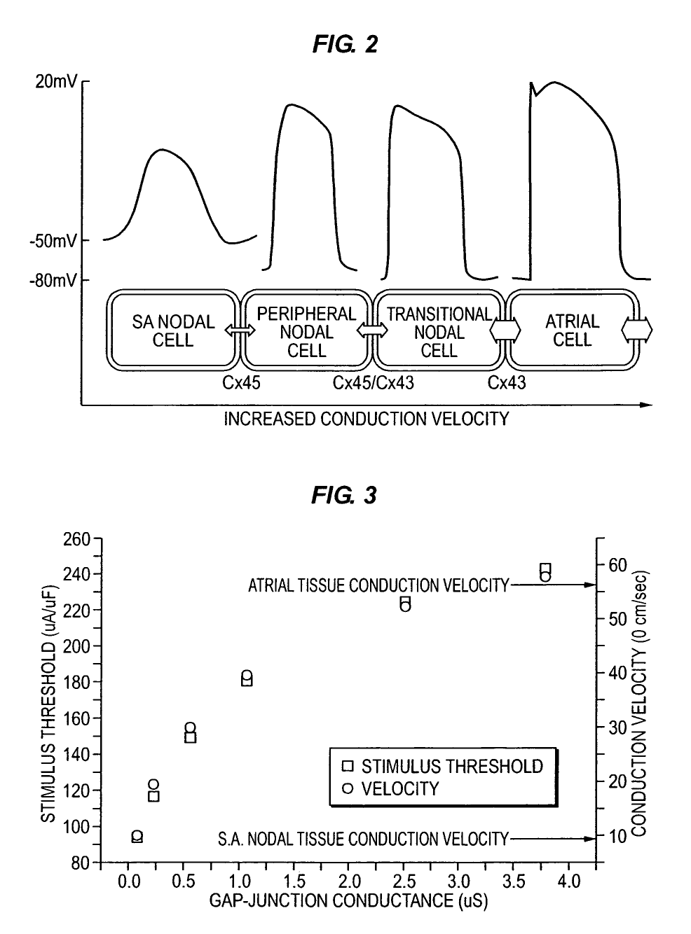

Because of regional differences in pacemaking, the SA node effectively has multiple pacemaker mechanisms. Although the periphery of an isolated SA node has the fastest intrinsic pacemaker activity, it is normally not the leading pacemaker site, because of suppression by the adjoining atrial muscle, which decreases (i.e., make more negative) the resting membrane potential. Such suppression is carried out, at least in part, by a regional presence or absence of connexins. See FIG. 2 illustrating a schematic diagram of the physiological gradient of conduction velocity partially responsible for the relative differences action potential diastolic potentials, amplitudes, and durations from the center of the SA node to the peripheral nodal cells and atrial muscle. Thus, careful biological regulation of Cx43 and Cx45 permit the SA node to function properly. See Boyett et al., J. Cardiovasc. Electrophysiol., (2003) 14: 104-106.

Also, the decreased conductivity between cells has been shown to reduce the current required to pace adjacent cells by creating less of a current sink, and therefore enhances pacing. See FIG. 3 depicting in silico modeling data demonstrating the influence of gap junction conductance and on current density stimulation thresholds and conduction velocity.

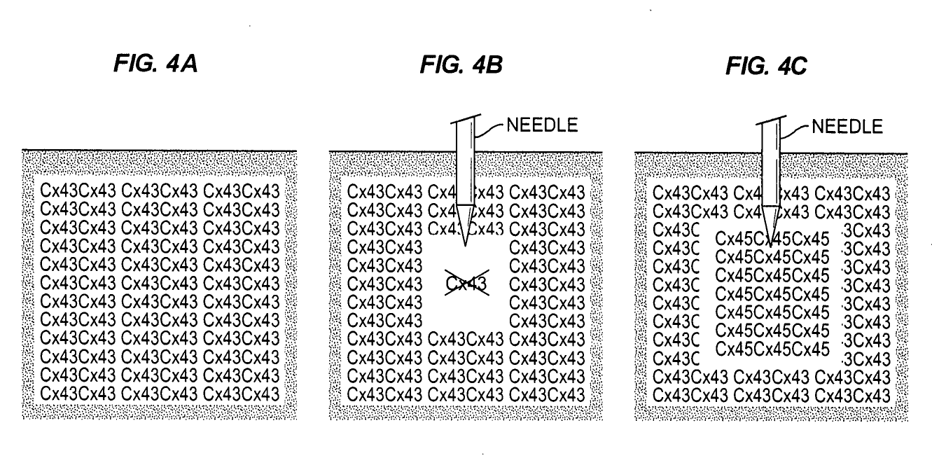

FIGS. 4 A-C show (A) normal Cx distribution in atrial (“A”) or ventricular (“V”) regions, (B) the suppression of the expression of the Cx43 protein by small interfering RNA, and (C) the Cx45 overexpression at relatively low levels, the latter of which may not be necessary. Expression of connexin proteins may be suppressed or induced, depending on the disorder and therapy.

Modulation of the expression of connexin 43 is different from producing cells that may have the capability of spontaneously depolarizing. More specifically, conduction velocity close to 0.03 m/sec is achieved by modulation of connexin expression. Downregulation or suppression of the expression of connexin 43 is often desired and, depending on the degree of Cx43 downregulation (e.g., complete downregulation), upregulation of the lesser conductive Cx45 isoform may be desirable in order to create SA nodal electrophysiology.

Partial electrical isolation of the recombinant cells via suppression of connexin 43 prevents re-entrant tachyarrhythmias that compromises the function of the recombinant cells. For example, under certain circumstances, modulating only the expression of Cx43 may prolong action potential duration and increase refractoriness—thereby compromising the propensity of re-entry arrhythmias. Furthermore, decoupling of the cells in a different nodal region may decrease the amount of HCN, I_{K1}, or beta-adrenergic overexpression needed for automaticity (i.e., decoupling may result in decreased current density thresholds). See FIG. 3.

Table 1 lists candidate siRNA sequences used to modulate the expression of connexin 43 protein in connection with the methods of the present invention.

TABLE 1

| | | | |
|---|---|---|---|
| | | Starting | |
| | Sequence | Position in | % G/C |
| Candidate siRNA Sequence | Region | AF151980 | Content |

| | | | |
|---|---|---|---|
| GACAAGGTTCAAGCCTACT (SEQ. ID NO. 1) | ORF | 34 | 47.37% |
| AGGTTCAAGCCTACTCAAC (SEQ. ID NO. 2) | ORF | 38 | 47.37% |
| GGTTCAAGCCTACTCAACT (SEQ. ID NO. 3) | ORF | 39 | 47.37% |
| GTTCAAGCCTACTCAACTG (SEQ. ID NO. 4) | ORF | 40 | 47.37% |
| CAGTCTGCCTTTCGTTGTA (SEQ. ID NO. 5) | ORF | 145 | 47.37% |
| AGTCTGCCTTTCGTTGTAA (SEQ. ID NO. 6) | ORF | 146 | 42.11% |
| TGCCTTTCGTTGTAACACT (SEQ. ID NO. 7) | ORF | 150 | 42.11% |
| CCTTTCGTTGTAACACTCA (SEQ. ID NO. 8) | ORF | 152 | 42.11% |
| TTGTAACACTCAGCAACCT (SEQ. ID NO. 9) | ORF | 159 | 42.11% |
| TCTTGTACCTGGCTCATGT (SEQ. ID NO. 10) | ORF | 269 | 47.37% |
| CTTGTACCTGGCTCATGTG (SEQ. ID NO. 11) | ORF | 270 | 52.63% |
| TTGTACCTGGCTCATGTGT (SEQ. ID NO. 12) | ORF | 271 | 47.37% |
| TGTACCTGGCTCATGTGTT (SEQ. ID NO. 13) | ORF | 272 | 47.37% |
| TCTATGTGATGCGAAAGGA (SEQ. ID NO. 14) | ORF | 290 | 42.11% |
| CTATGTGATGCGAAAGGAA (SEQ. ID NO. 15) | ORF | 291 | 42.11% |
| ACTTGAAGCAGATTGAGAT (SEQ. ID NO. 16) | ORF | 377 | 36.84% |
| CTTGAAGCAGATTGAGATA (SEQ. ID NO. 17) | ORF | 378 | 36.84% |
| GTTGCTGCGAACCTACATC (SEQ. ID NO. 18) | ORF | 450 | 52.63% |
| TTGCTGCGAACCTACATCA (SEQ. ID NO. 19) | ORF | 451 | 47.37% |
| TGCTGCGAACCTACATCAT (SEQ. ID NO. 20) | ORF | 452 | 47.37% |
| GCTGCGAACCTACATCATC (SEQ. ID NO. 21) | ORF | 453 | 52.63% |
| CTGCGAACCTACATCATCA (SEQ. ID NO. 22) | ORF | 454 | 47.37% |
| AACCTACATCATCAGTATC (SEQ. ID NO. 23) | ORF | 459 | 36.84% |
| ACCTACATCATCAGTATCC (SEQ. ID NO. 24) | ORF | 460 | 42.11% |
| TATCCTCTTCAAGTCTATC (SEQ. ID NO. 25) | ORF | 474 | 36.84% |
| GCTGATCCAGTGGTACATC (SEQ. ID NO. 26) | ORF | 510 | 52.63% |
| CTGATCCAGTGGTACATCT (SEQ. ID NO. 27) | ORF | 511 | 47.37% |
| TGATCCAGTGGTACATCTA (SEQ. ID NO. 28) | ORF | 512 | 42.11% |
| TCTATGGATTCAGCTTGAG (SEQ. ID NO. 29) | ORF | 527 | 42.11% |
| AGCTTGAGTGCTGTTTACA (SEQ. ID NO. 30) | ORF | 538 | 42.11% |
| CTGGTTACTGGCGACAGAA (SEQ. ID NO. 31) | ORF | 862 | 52.63% |
| TGGTTACTGGCGACAGAAA (SEQ. ID NO. 32) | ORF | 863 | 47.37% |
| GGTTACTGGCGACAGAAAC (SEQ. ID NO. 33) | ORF | 864 | 52.63% |
| GTTACTGGCGACAGAAACA (SEQ. ID NO. 34) | ORF | 865 | 47.37% |
| TTACTGGCGACAGAAACAA (SEQ. ID NO. 35) | ORF | 866 | 42.11% |
| TACTGGCGACAGAAACAAT (SEQ. ID NO. 36) | ORF | 867 | 42.11% |
| ACTGGCGACAGAAACAATT (SEQ. ID NO. 37) | ORF | 868 | 42.11% |
| CTGGCGACAGAAACAATTC (SEQ. ID NO. 38) | ORF | 869 | 47.37% |
| GCCGCAATTACAACAAGCA (SEQ. ID NO. 39) | ORF | 893 | 47.37% |
| CCGCAATTACAACAAGCAA (SEQ. ID NO. 40) | ORF | 894 | 42.11% |
| CCGATGATAACCAGAATTC (SEQ. ID NO. 41) | ORF | 1013 | 42.11% |
| CGATGATAACCAGAATTCT (SEQ. ID NO. 42) | ORF | 1014 | 36.84% |
| ATTGTGGACCAGCGACCTT (SEQ. ID NO. 43) | ORF | 1072 | 52.63% |

The subject invention provides novel nucleic acid compositions, oligonucleotides that interfere with the expression of connexin 43 proteins or biologically active fragments, homologs, or analogues thereof. Expression of the connexin 43 protein may be modulated by use of a construct, plasmid or vector that contains one of the oligonucleotides of the subject invention. Cells or cell lines may be transformed or transfected with such constructs to modulate the expression of the connexin 43 protein.

The methods of the subject invention may further utilize genes encoding for other proteins, mutant or fragment thereof, and/or genes used to suppress the expression of such protein. Genes encoding other connexin proteins such as connexin 45 may be used together with the oligonucleotide of the subject invention. Hence, the oligonucleotide may be delivered to the heart to suppress expression of connexin 43 in combination with a gene that encodes for and regulates the expression of connexin 45. The genes can be transfected into a cell in single or multiple constructs, plasmids or vectors.

Furthermore, the oligonucleotides of the subject invention may be used together with other biologically active agents including a variety of proteins and peptides to treat cardiac disorders. For example, it was recently observed that cells in and around the SA node do not express atrial natriuretic peptide (ANP). See Boyett et al., J. Cardiovasc. Electrophysiol., (2003) 14: 104-106. Therefore, if the method of the subject invention is to be applied to the native sinoatrial nodal region (to repair/revive nodal cells), an ANP suppressor gene may be used. In such embodiments, only cells that do not express ANP (nodal) cells transcribe the delivered genes.

Oligonucleotides of the present invention and siRNA may be designed and optimized in accordance with conventional methods. See e.g, Elbashir EMBO J. (2001) 20: 6877-6888; Bernstein et al., Nature (2001) 409:363-366. See also, Kitabwalla et al., N Engl J Med (2002) 347 (17): 1364-1367; Hutvagner et al., Curr Opin Genetics & Development (2002) 12:225-232; Sharp, Genes Dev (2001) 15: 485-490.

Suppression of the expression of connexin proteins has been shown to lead to ventricular arrhythmias. Van Rijen, H. V. et al, Slow Conduction and Enhanced Anisotropy Increase the Propensity for Ventricular Tachyarrhythmias In Adult Mice with Induced Deletion of Connexin43, 2004 Mar. 2; 109(8): 1048-55, Epub 2004 Feb. 16. Furthermore, viral based myocardial gene therapy approaches have been shown to alter cardiac function. Williams, M. L., Viral-Based Myocardial Gene Therapy Approaches to Alter Cardiac Function, Annu Rev Physiol. 2004; 66:49-75; Chaudhri, B. B., Gene Transfer In Cardiac Myocytes, Sugr Clin North Am. 2004 February; 84(1): 141-59, ix-x.

Modulation of connexin 43 with an oligonucleotide of the present invention may be combined with modulation of other genes or proteins. For example, the genomic DNA of connexin 45 or a connexin cDNA may be used. The genomic DNA or cDNA is often operably linked to a promoter that is normally associated with the connexin sequence (e.g., a promoter endogenous to the connexin gene) or that is heterologous to the connexin sequence (i.e., a promoter from a source other than the connexin sequence).

As noted above, connexin proteins are a family of homologous proteins found in connexins of gap junctions as homo- or heterohexameric arrays. Connexin proteins are the major gap junction protein involved in the electrical coupling of cells. Gap junctions regulate intercellular passage of molecules, including inorganic ions and second messengers, thus achieving electrical coupling of cells. Over 15 connexin subunit isoforms are known, varying in size between about 25 kDa and 60 kDa and generally having four putative transmembrane .quadrature.-helical spanners. Different connexins are specific for various parts of the heart. As noted above, connexin family proteins found in the cardiovascular system includes Cx37, Cx40, Cx43, and Cx45.

The connexin sequences suitable for use in connection with the methods of the subject invention may be based on the nucleotide sequences of any species (e.g., mammalian or non-mammalian (e.g., reptiles, amphibians, avian (e.g., chicken)), particularly mammalian, including human, rodent (e.g., murine or rat), bovine, ovine, porcine, murine, or equine, preferably rat or human) and can be isolated or produced from any source whether natural, synthetic, semi-synthetic or recombinant. Where the transfected cell is a human cell, or where the cardiac tissue into which the cell is to be implanted is human, the connexin is preferably a human connexin or derived from a human connexin.

In addition, modified or mutant connexin sequences may be used in combination with oligonucleotides together and/or other biologically active agents and transfected into cell lines. A connexin protein may be mutated in one of various ways known to one skilled in the art.

Methods of making constructs are well known to those skilled in the art. For example, constructs containing the connexin 43 gene are described by E1 Oakley et al, Ann. Thorac. Surg. (2001) 71:1724-33. The construct may contain any one of a variety of promoters. For example, the promoter may be a regulated promoter (e.g., inducible promoter), such as a tetracycline-regulated promoter, expression from which can be regulated by exposure to an exogenous substance (e.g., tetracycline). Another example of regulated promoter system is the lac operator repressor gene regulatory system to regulate mammalian promoters (Cronin, et. al., Genes Dev. 2001 Jun. 15, 15(12):1506-17). The promoter may be a strong promoter that functions in mammalian cells, such as a promoter from cytomegalovirus (CMV), mouse mammary tumor virus (MMTV), Rous sarcoma virus (RSV), lenti-virus or adenovirus. Exemplary promoters include the promoter from the immediate early gene of human CMV (Boshart et al., Cell 41:521 530, 1985) and the promoter from the long terminal repeat (LTR) of RSV (Gorman et al., Proc. Natl. Acad. Sci. USA 79:6777-6781, 1982).

Alternatively, the promoter used may be a strong general eukaryotic promoter such as the actin gene promoter. In one embodiment, the promoter used may be a tissue-specific promoter. For example, the promoter used in the construct may be a cardiac cell specific promoter, a myoblast specific promoter or an adult skeletal muscle cell specific promoter (Luo, et. al., Development 2001 February, 128(4):459-69; Lee, et. al., J. Thor. Card. Sur. 1999 July, 118(1):26-4, discussion 34-5). Primary cardiac myocytes from neonatal rats have been transfected with a reporter construct driven by the C promoter of rat acyl-coenzyme synthetase gene (Kanda, et al. Heart Vessels 2000, 15(4): 191-6) as well as alpha- and beta-cardiac myosin heavy chain gene promoters (James, et. al., Circulation 2000 Apr. 11, 101(14):1715-21).

A few commercial companies are now marketing construct and vectors that have the ability to control the expression of a transgene by placing a promoter activated by the presence of a pharmacological agent upstream of the transgene. For example, BD Biosciences markets a promoter system that activates the expression of a downstream gene when doxycycline is present. A tetracycline responsive element (TRE) that binds doxycycline is present within the promoter construct. When doxycycline is removed, transcription from the TRE is turned off in a highly dose-dependent manner. Addition of other specific promoter elements can enable promoters to respond to changes in the microenvironment within a tissue. See Discher et al., J. Biol. Chem. (1998) 273:26087-26093; Prentice et al., Cardiovascular Res. (1997) 35: 567-576; Webster, Gene Therapy (1999) 6:951-953; Webster, The Scientist (1999) 13:13.

In making a construct useful for transfection of the oligonucleotides of the subject invention, the sequence of the 5′ untranslated region, and further 5′ upstream sequences and 3′ downstream sequences of connexin 43, may be utilized for promoter elements, including enhancer binding sites, that provide for expression in tissues where the connexin polypeptide is normally expressed.

For transfection in eukaryotic cells, the construct typically contains at a minimum a eukaryotic promoter operably linked to the oligonucleotide. Other expression components or cassettes may be used and operably linked to the promoter. For example, the polyadenylation signal sequence may be selected from any of a variety of polyadenylation signal sequences known in the art. An exemplary polyadenylation signal sequence is the SV40 early polyadenylation signal sequence.

Hence, while the modulation of connexin expression may be accomplished using regulatory elements operably inserted into a construct comprising the oligonucleotide, other methods of regulating connexin expression may include genomic regulatory elements endogenous to the recombinant cell, or by the addition of compounds that modulate connexin expression (e.g., either at the time of or following implanting the recombinant cells.).

A construct may also include one or more introns, where appropriate, which can increase levels of expression of the DNA of interest, particularly where the DNA of interest is a cDNA (e.g., contains no introns of the naturally-occurring sequence). Any of a variety of introns known in the art may be used (e.g., the human-globin intron, which is inserted in the construct at a position 5′ to the DNA of interest).

The constructs of the invention may further include other biologically active agents, genetic components and/or expression cassettes. For example the serum response factor (SRF) gene has been shown to regulate transcription of numerous muscle and growth factor-inducible genes. Because SRF is not muscle specific, it has been postulated to activate muscle genes by recruiting myogenic accessory factors. Myocardin is a member of a class of muscle transcription factors, provides a mechanism whereby SRF can convey myogenic activity to muscle genes. (Wang, et. al., Cell. 2001 Jun. 29; 105(7):851-62).

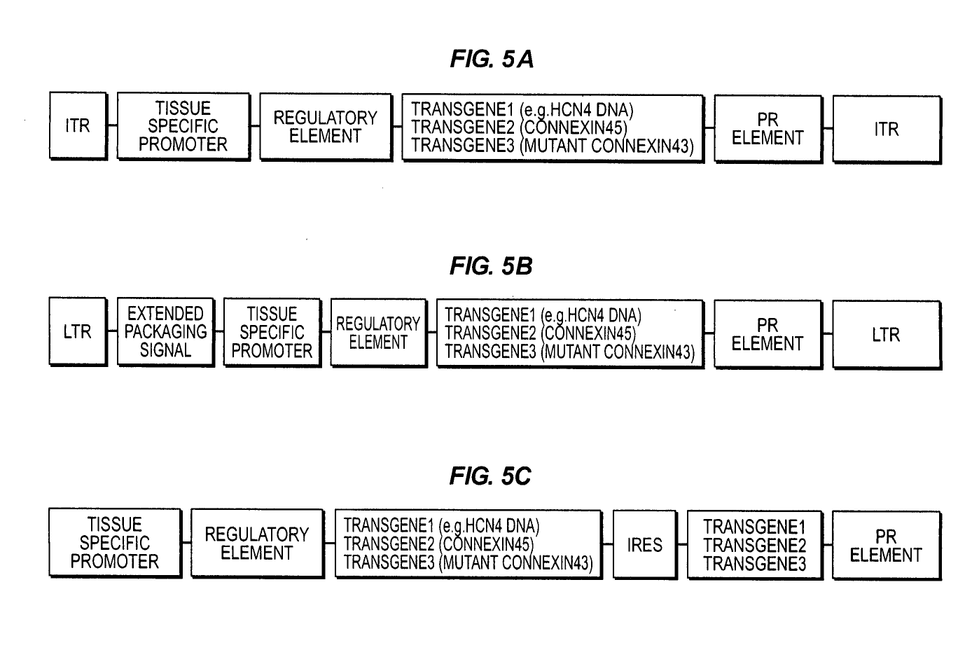

Exemplary constructs are shown in FIG. 5, wherein A-C depict three examples of viral vector expression cassettes containing Connexin 45 expressing as well as an If-generating vector: MV construct (A), Retroviral construct (B), and Adenoviral construct (shuttle plasmid) (C), and wherein:
ITR: inverted terminal repeats (AAV)
LTR: long terminal repeats (retrovirus)
Tissue specific promoter: myosin heavy chain, myosin light chain, others.
Regulatory Elements: Drug-responsive element which could suppress pacemaker activity pharmacologically (for example via tetracycline), others.
PR Element: Postregulatory element, could be used to enhance transgene expression.

There are numerous combinations of viral expression cassettes that can be generated in order to express at least two genes, i.e. a sequence for a connection protein together with the siRNA oligonucleotide sequences. Expression of these sequences can be driven either by their own separate promoters or by a single promoter with an IRES sequence separating the two genes. Examples include viral constructs such as AAV, retrovirus and adenovirus. Suitable viral vectors or contructs include, but are not limited to, adenoviruses, adeno-associated viruses, retroviruses, lentiviruses, semliki forst virus, herpes simplex viruses, vaccinia viruses, and combinations thereof.

Adenovirus viruses cause the common cold, have efficient entry into most cell types and can infect non-dividing cells. For gene therapy, this type of vector/construct is made replication-deficient by specifically deleting viral genes (e.g., E1, E2, E3 and/or E4). These genetically engineered vectors do not cause the common cold, although immune reactions to viral genes expressed in host cells can be observed.

As genetic constructs are made in vitro, they must be re-introduced into biological cells to use the internal machinery of these cells to produce multiple copies of the gene. Using this method, large amounts of protein can be produced and extracted and/or suppressed. Re-introduction of the genetic construct in a cell can be accomplished using a variety of methods, described below, including transfection, microinjection or nuclear transfer.

Hence, a cell suitable for use may be transformed with a vector, construct or plasmid. The recombinant cells can be cultured in vitro and utilized for transplantation into myocardium. The cells can be obtained from a fresh or frozen culture medium.

Suitable cells for use in connection with the present invention include muscle cells, stem cells (mesenchymal and hematopoietic), fibroblasts and cardiac cells. Cells may be endogenous cells (as obtained from a host) or from appropriate cultured cell lines. Hence the cells can be autologous, allogeneic, or xenogeneic (e.g., primate, pig, etc.). The cells may also be collected from a mammalian subject and/or via biopsy (e.g., muscle biopsy) allowing for autologous transplantation of the recombinant cell lines into host myocardium.

Cells particularly suitable for inducing the expression of connexin protein include any cell capable of coupling with a cardiomyocyte via connexin-mediated gap junctions, including stem cells (e.g., mesenchymal, hematopoietic, embryonic), cardiac cells, and the like, following genetic modification to provide for expression of a recombinant connexin (e.g., Cx43) in the cell. Skeletal muscle cells are particularly suitable for connexin expression. On the other hand, myoblasts and fibroblasts do not typically connect with myocardium.

Methods for introducing constructs into a mammalian cell include standard protocols known to those skilled in the art. See e.g., Davis, L. M., et al, Modulation of Connexin43 Expression: Effects on Cellular Coupling, J Cardiovasc Elecrophysiol 1995 February; 6(2): 103-14. Transfection of the genetic construct into a host cell may be achieved by exposing the genetic construct and the cells to electrical stimulus. This stimulus forms transient cellular pores on the membrane of the cell allowing the construct to diffuse into the cell. This method is generally used for tissue culture systems. Cells containing the genetic construct are selected and replicated forming multiple copies of the gene and subsequently the protein.

Cells that express or suppress connexin, for example, may be modified ex vivo and transplanted into any appropriate region of the patient's heart. A construct that provides for production of a desired gene may be introduced into cells that are propagated and cultured in vitro before and/or after transfection to increase the number of recombinant connexin-expressing cells available for transplantation into myocardial tissue.

Delivery of gene expression sequences (oligonucleotides) can also be achieved by non-viral vectors such as polymers, liposomes, and nanospheres, or by physical means such as electroporation. In addition, genetic material can be injected directly into the myocardium as described by Guzman et al., Circ. Res. (1993) 73:1202-1207.

Genetic material may also be delivered by the fluid delivery catheter described in commonly-assigned copending U.S. application Ser. No. 10/423,116, filed Apr. 23, 2003. As an example, FIG. 6 is a schematic diagram of the right side of a heart; similar to that shown in FIG. 1, wherein a guide catheter 90 is positioned for delivery of the genetic construct of the invention. A venous access site (not shown) for catheter 90 may be in a cephalic or subclavian vein and means used for venous access are well known in the art, including the Seldinger technique performed with a standard percutaneous introducer kit. Guide catheter 90 includes a lumen (not shown) extending from a proximal end (not shown) to a distal end 92 that slideably receives delivery system 80. Guide catheter 90 may have an outer diameter between approximately 0.115 inches and 0.170 inches and is of a construction well known in the art. Distal end 92 of guide catheter 80 may include an electrode (not shown) for mapping electrical activity in order to direct distal end 92 to an implant site near bundle of His 40. Alternatively a separate mapping catheter may be used within lumen of guide catheter 90 to direct distal end 92 to an implant site near bundle of His 40, a method well known in the art.

Gene therapy of the present invention can be used to heal a damaged SA node and/or in other areas of the heart. Connexin modulation alone can be used as a therapeutic treatment for arrhythmias such as tachyarrhythmias and other heart disorders.

Connexin suppression in the recombinat cells can be detected by such techniques as western blotting, utilizing antibodies specific for the connexin protein. Other methods for confirming the suppression of the expression of connexin in transformed cells may involve RT-PCR utilizing primers specific for connexin mRNA or immunofluorescence techniques on transformed cells in culture. The ability of a connexin to facilitate production of an electrical connection between a recombinant cell and a cardiomyocyte can be tested in an in vivo model.

The present invention may also be used in conjunction with a traditional pacemaker and/or defibrillator. In such embodiments, an implantable device can sense arrthymias via electrical sensing leads, then administer genetic therapy via a fluid delivery catheter lead. As genetic therapy is not instantaneous, a traditional implantable medical device, like a pacemaker, could be implanted as a safeguard. The various therapeutic uses of compounds, constructs or cells of the subject invention include uses in the treatment of a variety of different conditions in which modulation of the connexin 43 and/or 45 is desired. Exemplary diseases amenable to treatment by such methods include, but are not limited to, arrhythmias (ventricular tachycardia), congestive heart failure, heart block and the like. Any cardiac disease or disorder that would benefit from improved synchronized contraction is amenable to treatment with these methods of local enhancement or delivery of connexins, or biological equivalents thereof, to affect cellular gap junctions.

The transplantation of recombinant cells into the myocardium may also be administered to a subject by well-known surgical techniques for grafting tissue and/or isolated cells into a heart. In general, there are two methods for introducing the recombinant cells into the subject's heart tissue: 1) surgical, direct injection; or 2) percutaneous techniques as describe in U.S. Pat. No. 6,059,726 (Lee and Lesh, “Method for locating the AV junction of the heart and injecting active substances therein”).

The recombinant cells can be implanted into any area of the heart where conduction disturbances have occurred. The amount of recombinant cells to be transplanted is determined by the type of heart disease, the overall damage of myocardial tissue and the level of connexin modulation in the cells to be transplanted. Of particular interest with respect to cardiac stimulation aspects of the invention, the cells are delivered into a region of heart tissue to be stimulated, or to enhance propagation of such stimulation.

In certain embodiments, the recombinant cells are transplanted by percutaneous methods. If the site of the damaged heart tissue can be accurately determined in a subject by non-invasive diagnostic techniques, the recombinant connexin cells can be injected directly into the damaged myocardial tissue using general methods for percutaneous injections into cardiac muscle well known in the art, and further with respect to the novel, beneficial delivery embodiments provided herein. The amount of recombinant cells necessary to be therapeutically effective will vary with the type of disorder being treated as well as the extent of heart damage that has occurred.

Immunosuppressants may be used in conjunction of transplantation of the recombinant cells not derived from the host to minimize the possibility of graft rejection, e.g., allogeneic or xenogeneic cells.

The methods of the subject invention may also be utilized in combination with other cardiac therapies when appropriate. Drugs or biological agents used to treat certain types of conduction defects can be administered in combination with implanting recombinant cells into the damaged myocardium (e.g., prior to, during and/or after implantation). Biological agents that are suitable for use in combination therapy with connexin modulation methods include, but are not limited to, growth factors, polynucleotides encoding growth factors, angiogenic agents, calcium channel blockers, antihypertensive agents, antimitotic agents, inotropic agents, antiatherogenic agents, anti-coagulants, beta-blockers, anti-arrhythmic agents, antiinflammatory agents, vasodilators, thrombolytic agents, cardiac glycosides, antibiotics, antiviral agents, antifungal agents, agents that inhibit protozoans, antiarrhythmic agents (used for treatment of ventricular tachycardia), nitrates, angiotensin converting enzyme (ACE) inhibitors; brain natriuretic peptide (BNP); antineoplastic agents, steroids, and the like.

Connexin modulation may also be a supplemental procedure to coronary artery bypass grafting (CABG). Replacement of a non-functioning myocardial scar with functioning muscle together with revascularization improves myocardial performance more than revascularization (bypass surgery) alone. Transplantation of recombinant cells in conjunction with CABG provides for additive treatment during surgery by preventing the continued myocardial remodeling by reducing wall stress and ischemic burden. Additional surgical procedures to deliver the recombinant cells into the myocardium can be avoided by implanting the recombinant cells at the time of CABG surgery.

The effects of therapy can be monitored in a variety of ways. Generally for heart block disorders, an electrocardiogram (ECG) or holter monitor is utilized to determine the efficacy of treatment. The contraction of the heart occurs due to electrical impulses that are generated within the heart; an ECG is a measure of the heart rhythms and electrical impulses. Thus ECG is a very effective and non-invasive way to determine if therapy has improved or maintained, prevented, or slowed degradation of the electrical conduction in a subject's heart. The use of a holter monitor, a portable ECG that can be worn for long periods of time to monitor heart abnormalities, arrhythmia disorders, and the like, is also a reliable method to assess the effectiveness of therapy.

Electrophysiology tests involving percutaneous placement of catheters within the heart to assess the conduction properties of the heart, can also be used to assess therapy.

Where the condition to be treated with bioactive agent delivery is congestive heart failure, an echocardiogram or nuclear study can be used to determine improvement in ventricular function. Comparison of echocardiograms prior to and after the grafting of recombinant cells into myocardial tissue allows for reliable assessment of treatment.

The above methods for assessing the efficacy of therapy are only exemplary and are not meant to be limiting. Many appropriate assays for detecting synchronized coupling, (e.g., by monitoring cardiac function) are well known in the art and can be adapted for use. All patents and publications referenced herein are hereby incorporated by reference. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

What is claimed is:

1. An oligonucleotide comprising a sequence selected from the group consisting of SEQ ID NOS: 1-47.

2. A pharmaceutical composition comprising an oligonucleotide selected from the group consisting of SEQ ID NOS: 1-47 and a pharmaceutically acceptable carrier.

3. A construct useful for transfection or transformation of a cell comprising the oligonucleotide of claim 1.

4. A cell comprising the construct of claim 3.

5. A method of treating a cardiac disorder comprising administering to a mammal an oligonucleotide of claim 1.

6. The method of claim 5 wherein the cardiac disorder is arrhythmia.

7. The method of claim 5 further comprising administering to a mammal a gene encoding the connexin protein.

8. The method of claim 5 wherein the connexin protein is connexin 45.

9. The method of claim 5 further comprising administering to a mammal a biological activity agent.

10. The method of claim 6 wherein a fluid delivery catheter is used to administer the oligonucleotide to the mammal.

11. A method of correcting arrhythmia in a mammal by administering siRNA wherein said siRNA interferes with the expression of connexin 43 protein.

12. A kit comprising an oligonucleotide of claim 1 and a fluid delivery catheter.

13. The kit of claim 12, further comprising an electrical sensing lead.

14. The kit comprising an oligonucleotide of claim 1 and an implantable defibrillation device.


SEQUENCE LISTING (43 sequences)

SEQ ID NO: 1 - DNA, 19 bp, Artificial
  Small Interfering RNA
gacaaggttcaagcctact

SEQ ID NO: 2 - DNA, 19 bp, Artificial
  Small Interfering RNA
aggttcaagcctactcaac

SEQ ID NO: 3 - DNA, 19 bp, Artificial
  Small Interfering RNA
ggttcaagcctactcaact

SEQ ID NO: 4 - DNA, 19 bp, Artificial
  Small Interfering RNA
gttcaagcctactcaactg

SEQ ID NO: 5 - DNA, 19 bp, Artificial
  Small Interfering RNA
cagtctgcctttcgttgta

SEQ ID NO: 6 - DNA, 19 bp, Artificial
  Small Interfering RNA
agtctgcctttcgttgtaa

SEQ ID NO: 7 - DNA, 19 bp, Artificial
  Small Interfering RNA
tgcctttcgttgtaacact

SEQ ID NO: 8 - DNA, 19 bp, Artificial
  Small Interfering RNA
cctttcgttgtaacactca

SEQ ID NO: 9 - DNA, 19 bp, Artificial
  Small Interfering RNA
ttgtaacactcagcaacct

SEQ ID NO: 10 - DNA, 19 bp, Artificial
  Small Interfering RNA
tcttgtacctggctcatgt

SEQ ID NO: 11 - DNA, 19 bp, Artificial
  Small Interfering RNA
cttgtacctggctcatgtg

SEQ ID NO: 12 - DNA, 19 bp, Artificial
  Small Interfering RNA
ttgtacctggctcatgtgt

SEQ ID NO: 13 - DNA, 19 bp, Artificial
  Small Interfering RNA
tgtacctggctcatgtgtt

SEQ ID NO: 14 - DNA, 19 bp, Artificial
  Small Interfering RNA
tctatgtgatgcgaaagga

SEQ ID NO: 15 - DNA, 19 bp, Artificial
  Small Interfering RNA
ctatgtgatgcgaaaggaa

SEQ ID NO: 16 - DNA, 19 bp, Artificial
  Small Interfering RNA
acttgaagcagattgagat

SEQ ID NO: 17 - DNA, 19 bp, Artificial
  Small Interfering RNA
cttgaagcagattgagata

SEQ ID NO: 18 - DNA, 19 bp, Artificial
  Small Interfering RNA
gttgctgcgaacctacatc

SEQ ID NO: 19 - DNA, 19 bp, Artificial
  Small Interfering RNA
ttgctgcgaacctacatca

SEQ ID NO: 20 - DNA, 19 bp, Artificial
  Small Interfering RNA
tgctgcgaacctacatcat

SEQ ID NO: 21 - DNA, 19 bp, Artificial
  Small Interfering RNA
gctgcgaacctacatcatc

SEQ ID NO: 22 - DNA, 19 bp, Artificial
  Small Interfering RNA
ctgcgaacctacatcatca

SEQ ID NO: 23 - DNA, 19 bp, Artificial
  Small Interfering RNA
aacctacatcatcagtatc

SEQ ID NO: 24 - DNA, 19 bp, Artificial
  Small Interfering RNA
acctacatcatcagtatcc

SEQ ID NO: 25 - DNA, 19 bp, Artificial
  Small Interfering RNA
tatcctcttcaagtctatc

SEQ ID NO: 26 - DNA, 19 bp, Artificial
  Small Interfering RNA
gctgatccagtggtacatc

SEQ ID NO: 27 - DNA, 19 bp, Artificial
  Small Interfering RNA
ctgatccagtggtacatct

SEQ ID NO: 28 - DNA, 19 bp, Artificial
  Small Interfering RNA
tgatccagtggtacatcta

SEQ ID NO: 29 - DNA, 19 bp, Artificial
  Small Interfering RNA
tctatggattcagcttgag

SEQ ID NO: 30 - DNA, 19 bp, Artificial
  Small Interfering RNA
agcttgagtgctgtttaca

SEQ ID NO: 31 - DNA, 19 bp, Artificial
  Small Interfering RNA
ctggttactggcgacagaa

SEQ ID NO: 32 - DNA, 19 bp, Artificial
  Small Interfering RNA
tggttactggcgacagaaa

SEQ ID NO: 33 - DNA, 19 bp, Artificial
  Small Interfering RNA
ggttactggcgacagaaac

SEQ ID NO: 34 - DNA, 19 bp, Artificial
  Small Interfering RNA
gttactggcgacagaaaca

SEQ ID NO: 35 - DNA, 19 bp, Artificial
  Small Interfering RNA
ttactggcgacagaaacaa

SEQ ID NO: 36 - DNA, 19 bp, Artificial
  Small Interfering RNA
tactggcgacagaaacaat

SEQ ID NO: 37 - DNA, 19 bp, Artificial
  Small Interfering RNA
actggcgacagaaacaatt

SEQ ID NO: 38 - DNA, 19 bp, Artificial
  Small Interfering RNA
ctggcgacagaaacaattc

SEQ ID NO: 39 - DNA, 19 bp, Artificial
  Small Interfering RNA
gccgcaattacaacaagca

SEQ ID NO: 40 - DNA, 19 bp, Artificial
  Small Interfering RNA
ccgcaattacaacaagcaa

SEQ ID NO: 41 - DNA, 19 bp, Artificial
  Small Interfering RNA
ccgatgataaccagaattc

SEQ ID NO: 42 - DNA, 19 bp, Artificial
  Small Interfering RNA
cgatgataaccagaattct

SEQ ID NO: 43 - DNA, 19 bp, Artificial
  Small Interfering RNA
attgtggaccagcgacctt